(19) United States
(12) Patent Application Publication
(10) Pub. No.: US 2011/0190658 A1
(43) Pub. Date: Aug. 4, 2011

(54) PORTABLE SOUND SOURCE REPRODUCING APPARATUS FOR TESTING HEARING ABILITY AND METHOD USING THE SAME

(75) Inventors: Jun-il SOHN, Yongin-si (KR); Dong-wook KIM, Seoul (KR); Yoon-seo KOO, Yongin-si (KR); Hong-sig KIM, Yongin-si (KR); Kyoung-ho BANG, Seoul (KR); Jung-hak LEE, Seongnam-si (KR)

(73) Assignee: SAMSUNG ELECTRONICS CO., LTD., Suwon-si (KR)

(21) Appl. No.: 13/015,116

(22) Filed: Jan. 27, 2011

(30) Foreign Application Priority Data
Feb. 2, 2010 (KR) 10-2010-0009643

(51) Int. Cl. A61B 5/00

(52) U.S. Cl. 600/559

(57) ABSTRACT

A method of testing hearing ability using a sound source reproducing apparatus, the method comprises outputting at least one test sound representing at least one frequency band, acquiring a response of a testee to a degree of audibility of the at least one test sound, and testing hearing ability of the testee in the at least one frequency band corresponding to each of the at least one test sound, respectively, according to the response of the testee.

### CROSS-REFERENCE TO RELATED APPLICATIONS

This application claims priority to Korean Patent Application No. 10-2010-0009643, filed on Feb. 2, 2010, and all the benefits accruing therefrom under 35 U.S.C. §119, the content of which in its entirety is herein incorporated by reference.

### BACKGROUND

1. Field

The present disclosure generally relates to portable sound source reproducing apparatuses for testing hearing ability and methods of testing hearing ability using the apparatuses.

2. Description of the Related Art

Recently, the number of people who suffer from hearing loss has been increasing due to an increased use of personal audio apparatuses, an increase in the number of aged people, and an increasing number of noisy environments. A hearing test may be performed by observing a reaction of a testee who hears a sound, e.g., a pure tone whose frequency and amount thereof may vary.

### SUMMARY

Provided are portable sound source reproducing apparatuses for testing hearing ability and methods of testing hearing ability using the portable sound source reproducing apparatuses. Provided are embodiments of a computer-readable recording media including a program for executing the methods. A technical goal of the portable sound source reproducing apparatuses for testing hearing ability is not limited as above, and various other technical goals may also exist.

According to an aspect of the present invention, a method of testing hearing ability using a sound source reproducing apparatus includes outputting at least one test sound representing at least one frequency band frequency band, acquiring a response of a testee to a degree of audibility of the at least one test sound; and testing hearing ability of the testee in the at least one frequency band corresponding to each of the at least one test sound according to the response of the testee.

According to another aspect of the present invention, provided is a computer-readable recording medium including a program for executing the method.

According to another aspect of the present invention, a sound source reproducing apparatus includes: an output unit to output at least one test sound representing each frequency band; a user interface unit to receive a response of a testee to the degree of audibility of each of the at least one test sound output from the output unit; and a processor to test hearing ability of the testee in a frequency band corresponding to each of the at least one test sound according to the response of the testee provided through the user interface unit.

### BRIEF DESCRIPTION OF THE DRAWINGS

The above and other aspects, advantages and features of this disclosure will become more apparent by describing in further detail embodiments thereof with reference to the accompanying drawings, in which:

FIG. 1 is a block diagram of an embodiment of a sound source reproducing apparatus according to the present disclosure;

FIG. 2 illustrates frequency characteristics of Korean phonemes;

FIG. 3 illustrates frequency characteristics of English phonemes;

FIGS. 4A and 4B illustrate an embodiment of a user interface unit of the sound source reproducing apparatus of FIG. 1 which is used to output a test sound, according to the present disclosure;

FIGS. 5A through 5C illustrate an embodiment of a user interface unit of the sound source reproducing apparatus of FIG. 1 which is used to output a test sound, according to the present disclosure;

FIG. 6 is a graph illustrating a relationship between frequency and amplification gain; and

FIG. 7 is a flowchart illustrating an embodiment of a method of testing hearing ability of a testee using the sound source reproducing apparatus of FIG. 1, according to the present disclosure.

### DETAILED DESCRIPTION

The general inventive concept now will be described more fully hereinafter with reference to the accompanying drawings, in which various embodiments are shown. This invention may, however, be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

Hereinafter, embodiments of the present invention will be described in further detail with reference to the accompanying drawings.

Reference will now be made in detail to embodiments, examples of which are illustrated in the accompanying drawings, wherein like reference numerals refer to the like elements throughout.

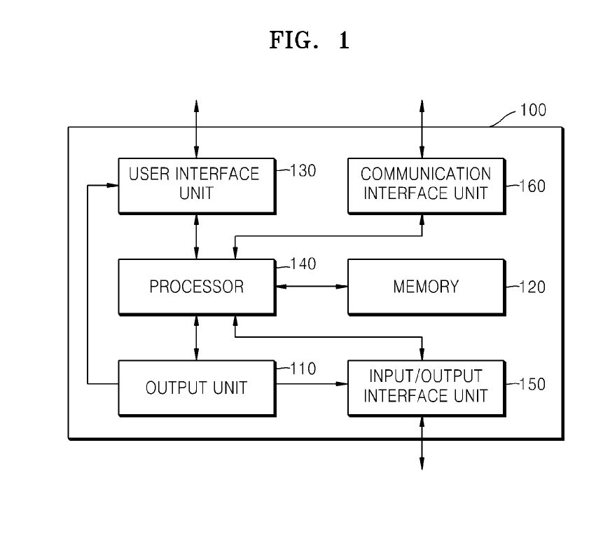

FIG. 1 is a block diagram of an embodiment of a sound source reproducing apparatus 100 according the present disclosure. Referring to FIG. 1, the sound source reproducing apparatus 100 includes an output unit 110, a memory 120, a user interface unit 130, a processor 140, an input/output interface unit 150, and a communication interface unit 160. The following explanation may be made by focusing on elements of the sound source reproducing apparatus 100 which are used to test hearing ability, display a result of the testing, and reproduce a sound source using the result of the testing. Accordingly, it would be understood by one of ordinary skill in the art that the sound source reproducing apparatus 100 may further include other various types of elements other than the elements illustrated in FIG. 1.

The sound source reproducing apparatus 100, which is portable by a testee whose hearing ability is to be tested, is an apparatus for reproducing a sound source or outputting a signal for reproducing the sound source. The term “portable” means that the sound source reproducing apparatus 100 is substantially small enough to be carried or worn by the testee. A sound source reproducing apparatus refers to an apparatus for generating an audible sound, which the testee can clearly hear, from data that is stored as a digital signal or an analog signal, or for generating a signal for generating the audible sound. Examples of the sound source reproducing apparatus include, but are not limited to, a mobile phone, a personal digital assistance (“PDA”), a moving picture experts group (“MPEG”) audio layer-3 (“MP3”) player, a compact disc (“CD”) player, and a portable media player.

The sound source reproducing apparatus 100 is an apparatus not only for generating an audible sound, which a testee can clearly hear, or outputting a signal for reproducing the audible sound but also for testing hearing ability. Hearing ability refers to a person's ability to perceive sound by detecting vibrations via an organ such as the ears, for example, and a hearing test refers to a test that measures a sensitivity of the ears. That is, the sound source reproducing apparatus 100 is an apparatus for measuring a degree of hearing ability as well as for reproducing a sound source or outputting a signal for reproducing the sound source.

A signal for reproducing a sound source refers to a signal for generating a sound substantially equated to the sound source. The output unit 110 outputs a signal for generating a sound. The signal for generating a sound is a waveform having a frequency phase and amplitude, for example. The signal is converted into a sound by being amplified by an amplifier, or by another sound source reproducing apparatus such as an earphone, for example, which is connected to the sound source reproducing apparatus 100, through the input/output interface unit 150. The sound source reproducing apparatus 100 may test hearing ability of a testee by referring to a degree of audibility of the sound generated in the aforesaid manner.

The output unit 110 may output a signal representing at least one syllable. In one embodiment, the output unit 110 may output a signal representing at least one speech sound, i.e., a voiced sound. The speech sound is formed of at least one syllable, for example, a monosyllable consisting of one syllable, or polysyllables such as a disyllable consisting of two syllables or a trisyllable consisting of three syllables, for example. A syllable is phonetically a minimal unit of pronunciation. In the Korean language, for example, a syllable consists of either a central vowel with consonants (e.g., [symbol] consists of [symbol] , ┐, and [symbol] ), a consonant vowel combination (e.g., [symbol] consists of [symbol] and ┐), or a vowel (e.g., [symbol] consists of [symbol] ).

In one embodiment, a user may allow a sound to be generated from a signal output from the output unit 110 by manipulating the user interface unit 130 of the sound source reproducing apparatus 100. The sound generated from the signal output from the sound source reproducing apparatus 100 stimulates an auditory organ of the user so as to allow the user to hear the sound. The output unit 110 may output an audio signal for generating a sound, and the user interface unit 130, e.g., a speaker, may directly generate the sound using the output audio signal. In one alternative embodiment, the output unit 110 may output an audio signal for generating a sound, the input/output interface unit 150 may transmit the audio signal to another sound source reproducing apparatus, e.g., an earphone such as a Bluetooth earphone, or a speaker connected to the sound source reproducing apparatus 100, for example, thereby the another sound reproducing apparatus may generate and reproduce the sound.

Accordingly, the output unit 110 allows a sound to be directly generated by the sound source reproducing apparatus 100 through the user interface unit 130 or to be generated by another sound source reproducing apparatus connected to the sound source reproducing apparatus 100 through the input/output interface unit 150. A signal output from the output unit 110 may be generated by the processor 140 based on data read from the memory 120. In one embodiment, the output unit 110 may be included in the processor 140, but is not limited thereto, and may be an independent chip separate from the processor 140.

A plurality of test sounds which are classified according to a plurality of frequency bands is stored in the memory 120. The plurality of test sounds includes syllables in the plurality of frequency bands within an audible frequency range. The audible frequency range refers to a periodic vibration whose frequency is audible to human ears. The audible frequency range is generally about 15 hertz (Hz) to about 20 kilohertz (KHz). In one embodiment, the audible frequency range may be divided into a plurality of frequency bands, and syllables consisting of phonemes included in the plurality of divided frequency bands may be grouped.

The memory 120 may include a storage medium. It would be understood by one of ordinary skill in the art that the memory 120 may include, but not limited to, at least one of a hard disk drive (“HDD”), a read only memory (“ROM”), a random access memory (“RAM”), a flash memory, and a memory card, for example.

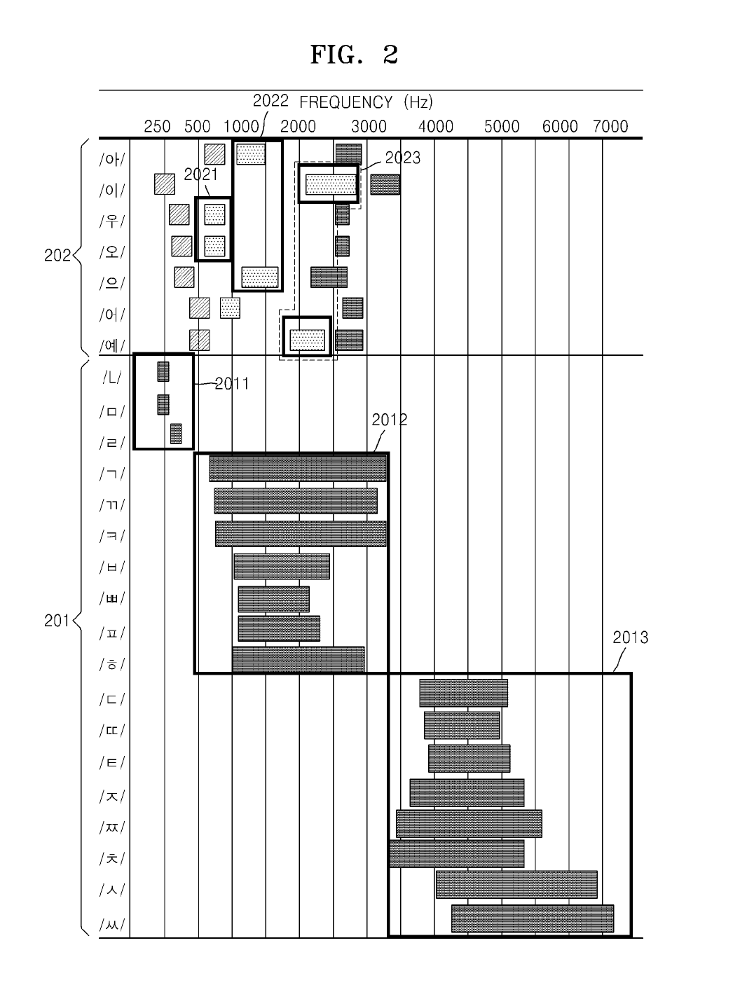

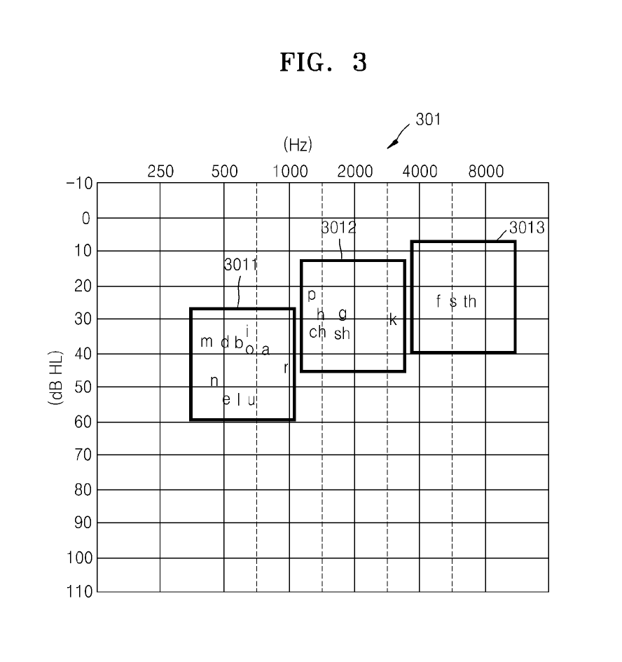

As described above, a syllable is a minimal unit of pronunciation of a language, and one syllable consists of at least one phoneme. As used in the field of phonetics, a phoneme refers to phonetically a minimal unit of phonological hierarchy. That is, a phoneme is a smallest unit of sound that distinguishes between meaningful utterances of speech. Korean phonemes include [symbol] [symbol] [symbol] [symbol] [symbol] and [symbol] , for example. Frequency characteristics of Korean phonemes and English phonemes will be explained later with reference to FIGS. 2 and 3.

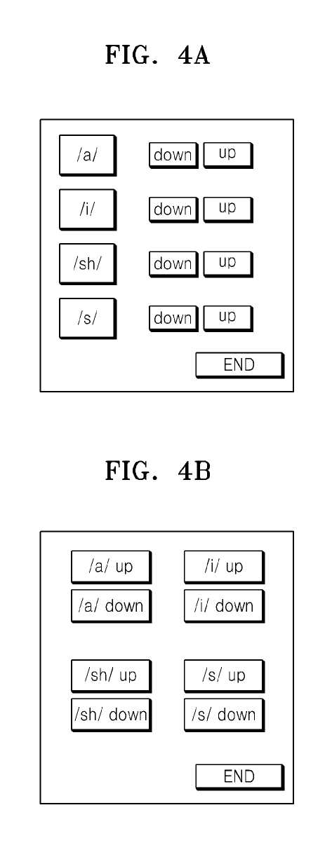

Groups of syllables divided according to frequencies are stored in the memory 120. The syllables included in the groups may be extracted from “phonetically balanced words.” As used herein, the term phonetically balanced word refers to a group of syllables including a range of phonemes that are overall balanced due to similar sound pressures generated when pronouncing the syllables. That is, a phonetically balanced word includes a wide frequency of sounds. The phonetically balanced word will be explained later with reference to FIG. 4.

A phonetically balanced word list 401 illustrated in FIG. 4 is stored in the memory 120. In one embodiment, the groups of the syllables divided according to the frequencies are stored in the memory 120. The syllables included in the groups may be extracted from the phonetically balanced word list 401 but are not limited thereto, and may not be extracted from the phonetically balanced word list 401.

The output unit 110 reads at least one syllable stored in the memory 120, and outputs a signal representing the at least one syllable. Assuming that the phonetically balanced word list 401 illustrated in FIG. 4 is stored in the memory 120, for example, a, the output unit 110 outputs a signal representing a plurality of syllables that are randomly extracted from syllables of the phonetically balanced word list 401. In one alternative embodiment, the output unit 110 extracts at least one syllable from each of the groups of the syllables divided according to the frequencies from the syllables of the phonetically balanced word list 401, and outputs a signal representing the at least one syllable. Hearing characteristics of the testee may be determined by observing the reaction of the testee to a sound generated from the signal output from the output unit 110.

When a response accuracy of the testee to syllables included in a group for a frequency band of about 1000 Hz to about 1300 Hz is 20%, for example, it may be determined that the testee has hearing characteristics of a low sensitivity to the frequency band. A signal for reproducing a sound source may be compensated for by setting an amplification gain of the frequency band to, for example, about 40 dB. In one alternative embodiment, a result of the testing of the hearing ability of the testee according to the hearing characteristics of the testee may be displayed using the user interface unit 130. In one embodiment, a sentence, such as “You have difficulty in hearing sounds in a high frequency band”, may be displayed to the testee through the user interface unit 130, or a graph showing a relationship between frequency and the degree of hearing loss of the testee may be displayed, for example. Accordingly, the sound source reproducing apparatus 100 may conveniently determine the hearing characteristics of the testee and compensation therefor may be applied to the sound source reproducing apparatus 100.

A signal output from the output unit 110 is reproduced as a sound using the user interface unit 130 or the input/output interface unit 150. In one embodiment, the sound source reproducing apparatus 100 of FIG. 1 may test the hearing ability of the testee using at least one syllable and thus may reduce a time taken to measure the reaction of the testee, compared to a method of measuring hearing ability of a testee using a pure tone. In one embodiment, the sound source reproducing apparatus 100 of FIG. 1 may prevent a reduction in the reliability of the testing which may be generated due to non-uniform pure tones.

In one embodiment wherein a hearing test is performed using a speech sound that is familiar to the testee, the hearing test using the speech sound takes a lot of time and the testee has to visit the location for conducting the hearing test. Since the sound source reproducing apparatus 100 is portable, such a hearing test may be substantially easily performed using the sound source reproducing apparatus 100.

The user interface unit 130 receives a response of the testee to the degree of audibility of a sound reproduced from the signal output by the output unit 110. The user interface unit 130 included in the sound source reproducing apparatus 100 receives an input signal from the testee, and displays output information to the testee. In one embodiment, the user interface unit 130 may include any of input/output devices such as a display panel, a mouse, a keyboard, an input button, a touch screen, a liquid crystal display (“LCD”) screen, and a monitor, for example. Accordingly, the user interface unit 130 may display a result of the testing of the hearing ability of the testee according to the hearing characteristics of the testee, and receive volume data from the testee. In one embodiment, the sound source reproducing apparatus 100 may obtain the response of the testee to the sound reproduced from the signal output from the output unit 110 by receiving the input signal input by the testee or recognizing the voice of the testee.

The testee hears a sound generated by the output unit 110 and inputs a response to the degree of audibility through the user interface unit 130. When a sound corresponding to a syllable “a” is reproduced at a predetermined volume level using the sound source reproducing apparatus 100, for example, the testee inputs a response to whether the testee can hear “a” at the predetermined volume level through the user interface unit 130. The degree of audibility refers to the degree of being audible by the ear. That is, the degree of audibility means whether the testee can clearly hear a test sound output from the sound source reproducing apparatus 100.

The user interface unit 130 may obtain a response to the degree of audibility from the testee using various user interfaces. In one embodiment, in order to measure the degree of comprehension of syllables, the degree of audibility may be measured by allowing the testee to directly input a syllable that the testee hears and determining whether the syllable input by the testee is the same as a syllable output from the sound source reproducing apparatus 100, for example. In one alternative embodiment, the degree of audibility may be measured by allowing the testee to select one syllable corresponding to a syllable output from the sound source reproducing apparatus 100 from among a plurality of syllables. In one alternative embodiment, the degree of audibility may be measured by allowing the testee to answer “Yes” or “No” with his/her voice or pushing a button, for example, indicating whether the testee can hear the syllable or not.

The aforesaid methods for obtaining a response to the degree of audibility from the testee using the sound source reproducing apparatus 100 are exemplary, and other various methods may be used.

The processor 140 controls overall functions of the sound source reproducing apparatus 100. The processor 140 controls the output unit 110, the memory 120, the user interface unit 130, the input/output interface unit 150, and the communication interface unit 160. The processor 140 tests the hearing ability of the testee based on the hearing characteristics indicating the degree of audibility in a frequency band corresponding to each test sound, according to the response input by the testee. That is, the processor 140 tests the hearing ability of a user using the method described above.

In one embodiment, the processor 140 may control the output unit 110 to output one signal, and the testee hears a sound generated from the signal output from the output unit 110 and inputs a response to the degree of audibility of the sound through the user interface unit 130, for example. The processor 140 determines hearing characteristics of the testee based on the response.

As described above, the testee inputs a response to the degree of audibility of each test sound through the user interface unit 130, and the processor 140 analyzes the response to determine hearing characteristics of the testee. That is, the processor 140 determines hearing characteristics of the testee in a frequency band represented by phonemes constituting syllables based on the response of the testee. When the testee has a low degree of audibility of syllables in a frequency band between about 600 Hz and about 800 Hz, that is, when the testee has a low response accuracy to the syllables included in the above frequency band, for example, it may be determined that the testee has difficulty in hearing sounds in the frequency band between about 600 Hz and about 800 Hz. Thus, a hearing test may be performed by determining the hearing ability of the testee based on a result of the determination.

In one embodiment, the processor 140 may correct a signal for reproducing a sound source using hearing characteristics of the testee. The processor 140 may correct a signal for reproducing a sound source by increasing a gain in a frequency range where the testee has difficulty in hearing according to the hearing characteristics of the testee to compensate for the signal for reproducing the sound source. The corrected signal is output by the output unit 110 and heard by the testee. In one embodiment, the processor 140 computes an algorithm for correcting an output signal by adjusting an amplification gain at a frequency of a sound source according to the hearing characteristics of the testee. The algorithm refers to any method used to convert between electrical signals and sound signals, such as a codec, for example. In one embodiment, the memory 120 may store the algorithm, and the sound source reproducing apparatus 100 may read the algorithm from the memory 120 to reproduce a sound source.

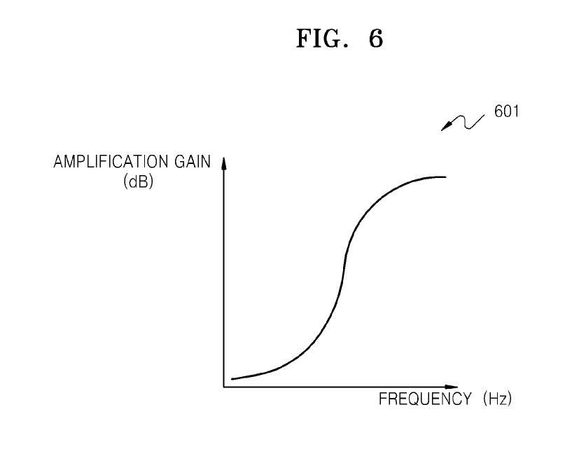

That is, the processor 140 computes an algorithm for correcting a signal for reproducing a sound source according to a result of the testing of the hearing ability of the testee, stores the algorithm in the memory 120, and applies the algorithm to all sound sources reproduced by the sound source reproducing apparatus 100. The algorithm may be a frequency versus amplification gain graph of a signal as shown in a frequency versus amplification gain graph 601 of FIG. 6. The processor 140 modifies a frequency versus amplification gain graph stored as a basic graph or generates a new frequency versus amplification gain graph according to the hearing characteristics of the testee, and stores the new frequency versus amplification gain graph in the memory 120.

In one embodiment, when the testee has a low degree of audibility of syllables in a frequency range between about 2000 Hz and about 3000 Hz, for example, the processor 140 computes an algorithm that sets an amplification gain of a signal in the frequency band between about 2000 Hz and about 3000 Hz to 40 decibel (dB). In the present embodiment, it would be obvious to one of ordinary skill in the art that the method is an example of algorithms for correcting a signal reproduced as a sound source. Accordingly, a method of correcting a signal reproduced as a sound source is not limited thereto, and other various methods according to the hearing characteristics of the testee may be used.

In one embodiment, the input/output interface unit 150 functions as an interface that connects the sound source reproducing apparatus 100 to another sound source reproducing apparatus so that a signal output from the output unit 110 is reproduced by the another sound source reproducing apparatus. That is, the input/output interface unit 150 transmits a signal output from the output unit 110 to another sound source reproducing apparatus connected to the sound source reproducing apparatus 100, and the another sound source reproducing apparatus reproduces the signal as a sound. In one embodiment, the input/output interface unit 150 may be a universal serial bus (“USB”) module or a head set jack provided in a mobile phone or an MP3 player corresponding to the sound source reproducing apparatus 100, for example, but is not limited thereto.

In a hearing test, hearing ability normally needs to be tested on both left and right ears, and thus another sound source reproducing apparatus for conducting the hearing test for both the ears, such as an ear phone, for example, may be used. Accordingly, the input/output interface unit 150 may function as an interface between the sound source reproducing apparatus 100 and any of electronic devices including other sound source reproducing apparatuses that are connected to the sound source reproducing apparatus 100 and reproduce as a sound a signal generated by the sound source reproducing apparatus 100.

The communication interface unit 160 allows data to be transmitted or received between the sound source reproducing apparatus 100 and external devices therethrough. In one embodiment, the communication interface unit 160 may not be included in the sound source reproducing apparatus 100 according to a user environment. In one embodiment when in the sound source reproducing apparatus 100 is a mobile phone, for example, general functions of the mobile phone, such as phone calls, transmission and reception of text messages, and Internet, may be performed by transmitting or receiving data through the communication interface unit 160.

Accordingly, the testee may substantially easily perform a hearing test using the sound source reproducing apparatus 100 and substantially immediately obtain a result of the hearing test. In one embodiment, by substantially immediately applying the result of the hearing test to the sound source reproducing apparatus 100, a degree of audibility of a speech sound may be substantially improved.

A volume level of a test sound initially output from the output unit 110 of the sound source reproducing apparatus 100 may be a volume level input by the testee through the user interface unit 130 or an initial volume level preset by the processor 140. The volume level input by the testee may be a volume level preferred by the testee. In one embodiment, the initial volume level preset by the processor 140 may be set to, for example, 40 to 60 dB, at which most testees feel substantially comfortable but the present embodiment is not limited thereto. In one embodiment, the initial volume level may also be adaptively set by previously testing the testee.

That is, since the sound source reproducing apparatus 100 is carried by the testee and the testee reproduces a sound source any time, a volume level of a sound preferred by the testee may be set. The sound source reproducing apparatus 100 may set a volume level of a test sound preferred by the testee and may conduct a hearing test based on the volume level.

Accordingly, the sound source reproducing apparatus 100 may test the hearing ability of the testee while being carried by the testee, display a result of the hearing test, and apply hearing characteristics of the testee to a sound source reproduced by the sound source reproducing apparatus 100 using the result of the hearing test. As a result, the testee may substantially easily hear the sound source to which the hearing characteristics of the testee are applied, without wearing any additional equipment for correcting the hearing ability, such as a hearing aid, for example.

Typical apparatuses for testing hearing ability are substantially difficult to use a result of a hearing test in testing hearing ability. However, the sound source reproducing apparatus 100 may directly use a result of a hearing test in testing hearing ability. Accordingly, the sound source reproducing apparatus 100 may perform a hearing test, and apply a result of the hearing test to any subsequent sound sources reproduced by the sound source reproducing apparatus 100.

FIG. 2 illustrates frequency characteristics of Korean phonemes. Referring to FIG. 2, Korean phonemes are divided into consonants and vowels and the consonants and the vowels are divided into a plurality of groups according to frequency ranges.

Referring to FIG. 2, a consonant list 201 includes consonants that are divided into three groups according to frequency ranges. A group 2011 includes consonants in a frequency range from about 220 Hz to about 500 Hz, a group 2012 includes consonants in a frequency range from about 650 Hz to about 3300 Hz, and a group 2013 includes consonants in a frequency range from about 3300 Hz to 7500 Hz. The consonants included in the group 2011 include [symbol] the consonants included in the group 2012 include [symbol] [symbol] and [symbol] and the consonants included in the group 2013 include [symbol] [symbol] [symbol] and [symbol] .

A vowel list 202 includes vowels according to frequency ranges. Referring to the vowel list 202, one vowel includes three formant frequencies. A formant frequency refers to a peak frequency having higher energy in a frequency spectrum which is obtained by integrating a time versus sound level (dB) curve of a vowel with a frequency axis. Referring to the vowel list 202, three formant frequencies constituting one vowel may be termed a first formant, a second formant, and a third formant sequentially from a low frequency band. As shown in the vowel list 202, since frequency bands of the first, the second, and the third formants are similar, vowels may be divided into three groups according to frequency bands of second formants. A group 2021 includes vowels whose second formants are in a frequency range from about 600 Hz to about 800 Hz, a group 2022 includes vowels whose second formants are in a frequency range from about 1000 Hz to about 1300 Hz, and a group 2023 includes vowels whose second formants are in a frequency range from about 2000 Hz to about 3000 Hz. The vowels included in the group 2021 include [symbol] and [symbol] the vowels included in the group 2022 include [symbol] and [symbol] and the vowels included in the group 2023 include [symbol] and [symbol] .

In one embodiment, after a hearing test, when the testee has a low degree of audibility of the consonants included in the group 2013 and fails to clearly distinguish the vowel [symbol] from the vowel [symbol] (or the vowel [symbol] from [symbol] ), for example, it may be determined that the testee has hearing loss in the frequency band from about 1000 Hz to about 1300 Hz that is the frequency range of the group 2022, and in the frequency band from about 3300 Hz to about 7500 Hz that is the frequency range of the group 2013.

FIG. 3 illustrates frequency characteristics of English phonemes. Referring to FIG. 3, English phonemes are divided into a plurality of groups according to frequency ranges.

Referring to FIG. 3, a phoneme list 301 includes phonemes that are divided into three groups according to frequency ranges. A group 3011 includes phonemes in a low frequency band from about 300 Hz to about 1200 Hz, a group 3012 includes phonemes in an intermediate frequency band from about 1200 Hz to about 3800 Hz, and a group 3013 includes phonemes in a high frequency band from about 3800 Hz to about 8000 Hz.

The phonemes included in the group 3011 include /m/, /d/, /b/, /i/, /o/, /a/, /n/, /e/, /l/./u/, and /r/, the phonemes included in the group 3012 include /p/, /h/, /g/, /k/, /ch/, and /sh/, and the phonemes included in the group 3013 include /f/, /s/, and /th/.

Accordingly, by referring to the frequency characteristics of the Korean phonemes and the English phonemes respectively illustrated in FIGS. 2 and 3, hearing characteristics of the testee according to a result of the hearting test may be determined.

In one embodiment, English monosyllables /a/ and /i/ are used as test sounds in a low frequency band, an English monosyllable /sh/ is used as a test sound in an intermediate frequency band, and an English monosyllable /s/ is used as a test sound in a high frequency band, but are not limited thereto, and the language, types, and number of monosyllables may vary in various ways. In one embodiment, combinations of Korean phonemes and English phonemes may be used as test sounds, for example.

FIGS. 4A and 4B illustrate an embodiment of the user interface unit 130 of the sound source reproducing apparatus 100 of FIG. 1 which is used to output a test sound, according to the present disclosure. In one embodiment, referring to FIG. 4A, the user interface unit 130 may include a touch screen on which monosyllable buttons (e.g., /a/, /i/, /sh/, and /s/), and volume down and up buttons are separately displayed as shown in FIG. 4A. In one alternative embodiment, the user interface unit 130 may include a touch screen on which monosyllable buttons each coupled with volume up and down buttons, e.g., /a/up, /i/up, /sh/up, /s/up, /a/down, /i/down, and /sh/down, /s/down, are displayed as shown in FIG. 4B.

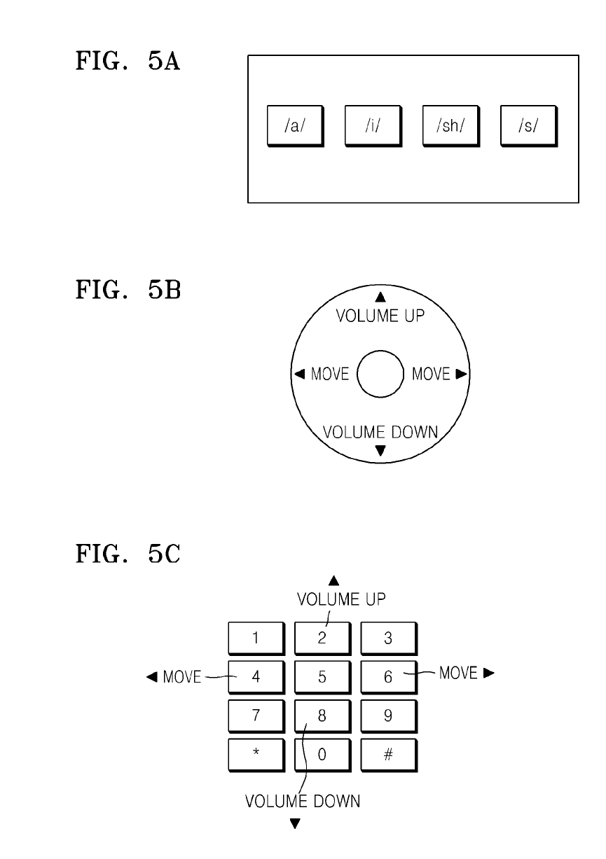

FIGS. 5A through 5C illustrate an embodiment of the user interface unit 130 of the sound source reproducing apparatus 100 of FIG. 2 which is used to output a test sound, according to the present disclosure. The user interface unit 130 may include a screen on which monosyllable buttons /at, /i/, /sh/, and /s/ are displayed, as shown in FIG. 5A, and various types of key panels including rightward and leftward buttons and volume up and down buttons are displayed, as shown in FIGS. 5B and 5C.

FIG. 6 is a graph illustrating a relationship between frequency and amplification gain. Referring to FIG. 6, a frequency versus amplification gain graph 601, which shows a change in an amplification gain as a frequency increases, may be applied to the testee who has difficulty in hearing in a high frequency band. That is, since an amplification gain of a sound in a low frequency band is low and an amplification gain of a sound in a high frequency band is high, the testee may hear a sound in a high frequency band, which the testee has difficulty in hearing, at a higher volume level.

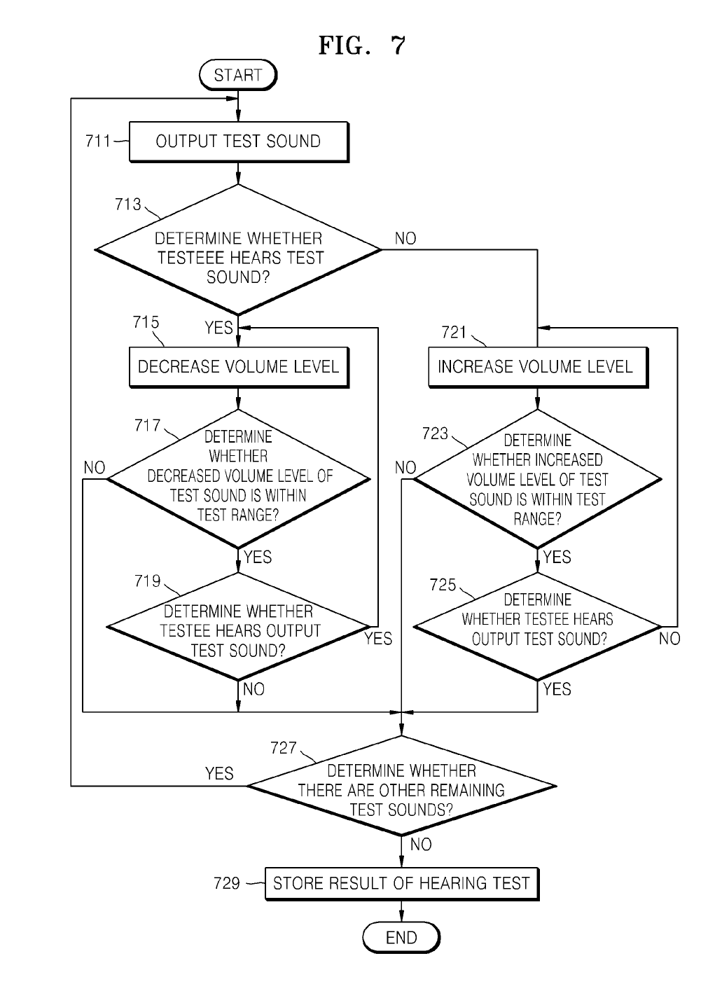

FIG. 7 is a flowchart illustrating an embodiment of a method of testing hearing ability of a testee using the sound source reproducing apparatus 100 of FIG. 1, according to the present disclosure. Referring to FIG. 7, the method includes operations sequentially performed using the sound source reproducing apparatus 100 of FIG. 1. Accordingly, although not made, descriptions already made for the sound source reproducing apparatus 100 of FIG. 1 may be also applied to the method.

In operation 711, one test sound from among a plurality of test sounds is output. In one embodiment, the test sounds may be output in an order that is selected by a user through the user interface unit 130, or may be automatically output in an order that is preset by the processor 140. In the present embodiment, each of the test sounds may include at least one monosyllable representing each of a low frequency band, an intermediate frequency band, and a high frequency band in an audible frequency range. In one alternative embodiment, sentences each consisting of disyllables, trisyllables, or polysillables according to frequency bands may be used as the test sounds. In one embodiment, the audible frequency range may be divided into frequency bands and at least one test sound may correspond to each of the frequency bands. In one embodiment, an initially output test sound may be output at an initial volume level preset by the processor 140, or may be output based on volume information input by the testee through a volume setting button of the user interface unit 130. In one embodiment, an initial volume level may range from about 40 dB to about 60 dB which is typically regarded as a substantially comfortable volume level, but the present embodiment is not limited thereto. The test sounds may be reproduced as a sound through the user interface unit 130 of the sound source reproducing apparatus 100, or through another sound source reproducing apparatus connected through the input/output interface unit 150 to the sound source reproducing apparatus 100.

In operation 713, it is determined whether the test sound output in operation 711 is heard by the testee using a response of the testee to the test sound output in operation 711. The response of the testee may be obtained in such a manner that the testee presses a button of the user interface unit 130, or the testee inputs the response with his/her voice. In one embodiment, the response of the testee may be obtained by allowing the testee to answer “Yes” or “No”, indicating whether the testee can hear, to a question such as “Can you hear?”, for example, displayed on a screen with a button or his/her voice.

In one embodiment, when it is determined in operation 713 that the testee hears the test sound, the method proceeds to operation 715. In operation 715, a volume level of the test sound is decreased by a predetermined unit by manipulating a volume setting button of the user interface unit 130, e.g., a volume down button. In the present embodiment, the predetermined unit may be set to, for example, 5 dB, but the present embodiment is not limited thereto and the predetermined unit may be appropriately set by considering test accuracy and test time. In operation 717, it is determined whether the decreased volume level of the test sound is within a test range. When it is determined in operation 717 that the decreased volume level of the test sound is within the test range, the test sound is output at the decreased volume level. When it is determined in operation 717 that the decreased volume level of the test sound exceeds the test range, the method proceeds to operation 727.

In operation 719, it is determined whether the test sound output in operation 717 is heard by the testee using the response of the testee to the test sound output in operation 717. When it is determined in operation 719 that the test sound is heard by the testee, the method returns to operation 715 in which the decreased volume level of the test sound is further decreased by manipulating the volume down button of the user interface unit 130. When it is determined in operation 719 that the test sound is not heard by the testee, however, the method proceeds to operation 727. In operation 727, it is determined whether there are other remaining test sounds. Operations 715 through 719 are repeatedly performed until the decreased volume level of the test sound exceeds the test range.

When it is determined in operation 713 that the test sound is not heard by the testee, however, the method proceeds to operation 721. In operation 721, the volume level of the test sound is increased by a predetermined unit by manipulating a volume setting button the user interface unit 130, e.g., a volume up button. In the present embodiment, the predetermined unit may be set to 5 dB, for example, but the present embodiment is not limited thereto and the predetermined unit may be appropriately set by considering test accuracy and test time. In operation 723, it is determined whether the increased volume level of the test sound is within the test range. When it is determined that the increased volume level of the test sound is within the test range, the test sound is output. When it is determined that the increased volume level of the test sound exceeds the test range, the method proceeds to operation 727.

In operation 725, it is determined whether the test sound output in operation 723 is heard by the testee using a response of the testee to the test sound output in operation 723. When it is determined in operation 725 that the test sound is not heard by the testee, the method returns to operation 721 in which the volume level of the test sound is increased further by manipulating the volume up button of the user interface unit 130. When it is determined in operation 725 that the test sound is heard by the testee, however, the method proceeds to operation 727. In operation 727, it is determined whether there are other remaining test sounds. Operations 721 through 725 are repeatedly performed until the increased volume level of the test sound exceeds the test range.

In operation 727, it is determined whether there are other remaining test sounds. When it is determined in operation 727 that all test sounds are used test, the method proceeds to operation 729. In operation 729, a result of the hearing test is stored in the memory 120. When it is determined in operation 727 that there are other remaining test sounds, the method returns to operation 711.

Hearing ability of the testee is tested based on a result of operations 713, 719, or 725, that is, information about whether the test sound in each frequency band is heard, or the degree of audibility of a frequency band corresponding to each test sound which is determined by a lowest volume level at which the test sound in each frequency band is audible. A signal for reproducing a sound source may be corrected by applying the result of the hearing test of the testee to the sound source reproducing apparatus 100. In one embodiment, the result of the hearing test of the testee may be displayed on the user interface unit 130.

The sound source reproducing apparatus 100 may include one or a plurality of the processors 140. The elements of the sound source reproducing apparatus 100 may correspond to the processor 140 or may respectively correspond to the plurality of processors 140. The processor 140 may include an array of logic gates, or a combination of a typical micro-processor and a memory in which a program that may be executed in the typical micro-processor is stored.

As described above, hearing characteristics of a testee may be easily tested using the sound source reproducing apparatus 100 and a result of a hearing test may be displayed on or applied to the sound source reproducing apparatus 100.

Accordingly, a reliable hearing test may be conveniently and quickly performed using a portable sound source reproducing apparatus using sounds of words which are familiar to a testee.

Embodiments of the present invention may be embodied in a general purpose digital computer by running a program from a computer-readable medium. Data used in the method may be recorded using various types of computer-readable recording mediums. Examples of the computer-readable mediums include storage media such as magnetic storage media, e.g., read only memories (“ROMs”), floppy discs, or hard discs, optically readable media, e.g., compact disk-read only memories (“CD-ROMs”), or digital versatile disks (“DVDs”).

While the portable source reproducing apparatus 100 for testing hearing ability has been particularly shown and described with reference to exemplary embodiments thereof, it will be understood by those of ordinary skill in the art that various changes in form and detail may be made therein without departing from the spirit and scope of the descriptions as defined by the following claims. The methods should be considered in a descriptive sense only and not for purposes of limitation. Therefore, the scope of the sound source reproducing apparatus 100 is defined not by the detailed description but by the appended claims, and all differences within the scope will be construed as being included in the sound source reproducing apparatus 100.

What is claimed is:

1. A method of testing hearing ability using a sound source reproducing apparatus, the method comprising:
outputting at least one test sound which represents at least one frequency band;
acquiring a response of a testee to a degree of audibility of the at least one test sound; and
testing hearing ability of the testee in the at least one frequency band corresponding to each of the at least one test sound, respectively, according to the response of the testee.

2. The method of claim 1, wherein the outputting of the at least one test sound comprises outputting at least one monosyllable which represents at least one of a low frequency band, an intermediate frequency band, and a high frequency band.

3. The method of claim 1, wherein the outputting of the at least one test sound comprises outputting the at least one test sound by increasing or decreasing a volume level of the at least one test sound according to the response of the testee to a degree of audibility of a test sound initially output in the acquiring of the response of the testee.

4. The method of claim 3, wherein the outputting of the at least one test sound comprises outputting another test sound when the increased or decreased volume level of the at least one test sound exceeds a test range.

5. The method of claim 1, wherein the outputting of the at least one test sound comprises outputting the at least one test sound in at least one of an order that is selected by the testee and an order that is automatically selected.

6. The method of claim 1, further comprising displaying a result of the testing of the hearing ability of the testee.

7. The method of claim 1, further comprising correcting a signal which reproduces a sound source using hearing characteristics of the testee.

8. The method of claim 7, wherein the correcting of the signal comprises correcting an amplification gain in the at least one frequency band according to the hearing characteristics of the testee.

9. The method of claim 1, wherein the acquiring of the response of the testee comprises acquiring the response of the testee based on information about whether the at least one test sound in the at lease one frequency band is heard or a lowest volume level at which the at least one test sound in the at lease one frequency band is audible.

10. The method of claim 1, wherein the acquiring of the response of the testee comprises acquiring the response of the testee to a degree of audibility of a test sound output from another sound source reproducing apparatus connected to the sound source reproducing apparatus.

11. A non-transitory computer-readable recording medium having embodied thereon a program for executing a method of testing hearing ability using a sound source reproducing apparatus, the method comprising:
outputting at least one test sound which represents at least one frequency band;
acquiring a response of a testee to a degree of audibility of the at least one test sound; and
testing hearing ability of the testee in the at least one frequency band corresponding to each of the at least one test sound according to the response of the testee.

12. A sound source reproducing apparatus comprising:
an output unit to output at least one test sound which represents at least one frequency band;
a user interface unit which receives a response of a testee to a degree of audibility of each of the at least one test sound output from the output unit; and
a processor which tests hearing ability of the testee in the at least one frequency band corresponding to each of the at least one test sound according to the response of the testee provided through the user interface unit.

13. The sound source reproducing apparatus of claim 12, further comprising a memory which stores at least one monosyllable which represents at least one of a low frequency band, an intermediate frequency band, and a high frequency band,
wherein the output unit outputs the at least one monosyllable as the at least one test sound.

14. The sound source reproducing apparatus of claim 12, wherein the user interface unit receives a signal that increases and decreases a volume level of the at least one test sound, and
wherein the processor tests hearing ability of the testee using a response of the testee which is obtained using information about whether the at least one test sound is heard at least one of the increased volume level, the decreased volume level, and a lowest level at which the at least one test sound corresponding to the at least one frequency band is audible.

15. The sound source reproducing apparatus of claim 12, wherein the user interface unit comprises a touch screen, and monosyllable buttons and a volume up button and a down button are displayed on the touch screen.

16. The sound source reproducing apparatus of claim 12, wherein the user interface unit comprises a screen and a key panel, and monosyllable buttons are displayed on the screen and a rightward button, a leftward button , a volume up button, and a volume down button are displayed on the key panel.

17. The sound source reproducing apparatus of claim 12, wherein the user interface unit displays a result of the testing of the hearing ability of the testee according to hearing characteristics of the testee.

18. The sound source reproducing apparatus of claim 12, wherein the processor corrects a signal which reproduces a sound source using hearing characteristics of the testee, and the output unit outputs the corrected signal.

19. The sound source reproducing apparatus of claim 18, wherein the processor computes an algorithm which corrects the signal using an amplification gain in the at least one frequency band according to the hearing characteristics of the testee, a memory unit stores the computed algorithm, and the output unit outputs the corrected signal obtained using the algorithm stored in the memory unit.

20. The sound source reproducing apparatus of claim 12, further comprising an input/output interface unit which transmits a signal output from the output unit to another sound source reproducing apparatus connected to the sound source reproducing apparatus,
wherein the user interface unit acquires a response of the testee to a degree of audibility of a sound output from the another sound source reproducing apparatus.
